(19) United States
(12) Patent Application Publication
(10) Pub. No.: US 2023/0338294 A1
(43) Pub. Date: Oct. 26, 2023

(54) NOVEL PHARMACEUTICAL FORMULATION FOR C-MET INHIBITOR

(71) Applicant: APOLLOMICS INC., Foster City, CA (US)

(72) Inventors: Sanjeev REDKAR, California, CA (US); Thomas STUMPFIG, Oregon, OR (US); Anne MUSKE-DUKES-DRIGGS, Oregon, OR (US); Thomas REYNOLDS, Oregon, OR (US); Thurman Russell FALK, Oregon, OR (US); Mike Tso-Ping LI, California, CA (US); Prema VIJAYAKUMAR, California, CA (US)

(21) Appl. No.: 17/997,126

(22) PCT Filed: Apr. 24, 2021
(86) PCT No.: PCT/US2021/029022

§ 371 Date: Oct. 26, 2022

Related U.S. Application Data
(60) Provisional application No. 63/015,675, filed on Apr. 26, 2020.

(51) Int. Cl. A61K 9/20; A61K 31/5025

(52) U.S. Cl. CPC A61K 9/2054; A61K 31/5025

(57) ABSTRACT

The present disclosure provides a pharmaceutical oral dosage form. In one embodiment, the dosage form comprises a formulation comprising an active pharmaceutical ingredient (API) and a polymer, wherein said API is a compound inhibiting c-Met tyrosine kinase.

### CROSS-REFERENCE TO RELATED APPLICATIONS

This application claims priority to U.S. provisional application 63/015,675, filed Apr. 26, 2020, the disclosure of which is incorporated herein by reference.

### FIELD OF THE INVENTION

The present disclosure relates generally to the fields of pharmaceutical formulation. In particular, the disclosure relates to dosage form of certain compounds that inhibit c-Met tyrosine kinase receptor.

### BACKGROUND

The Hepatocyte Growth Factor Receptor, also named as c-Met, is a receptor tyrosine kinase that has been shown to be over-expressed in a variety of malignancies, such as Small Cell Lung Cancer (SCLC) and NSCLC (Olivero et al., Br J Cancer, 74: 1862-8 (1996) and Ichimura et al., Jpn J Cancer Res, 87:1063-9 (1996)).

Inhibitors specifically against c-Met represent an attractive novel targeted therapeutic approach. APL-101 (see US20150218171), also known as PLB1001 and bozitinib, is a highly selective c-Met inhibitor that is currently in clinical trial as an anti-cancer agent. There is a need to develop new formulations and dosage forms for APL-101 to increase its bioavailability.

### SUMMARY

The present disclosure in one aspect provides a pharmaceutical composition. In one embodiment, the pharmaceutical composition comprises a formulation comprising an active pharmaceutical ingredient (API) and a polymer, wherein said API is a compound inhibiting c-Met tyrosine kinase.

In certain embodiments, the API is a compound of the following formula or a pharmaceutically acceptable salt thereof or a hydrate thereof:

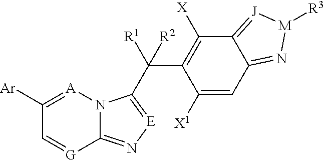

wherein:
R¹ and R² are independently hydrogen or halogen;
X and X¹ are independently hydrogen or halogen;
A and G are independently CH or N, or CH=G is replaced with a sulfur atom;
E is N;
J is CH, S or NH;
M is N or C;
Ar is aryl or heteroaryl, optionally substituted with 1-3 substituents independent selected from: C₁₋₆alkyl, C₁₋₆alkoxyl, halo C₁₋₆alkyl, halo C₁₋₆alkoxy, C₃₋₇cycloalkyl, halogen, cyano, amino, —CONR⁴R⁵, —NHCOR⁶, —SO₂NR⁷R⁸, C₁₋₆alkoxyl-, C₁₋₆alkyl-, amino-C₁₋₆alkyl-, heterocyclyl and heterocyclyl-C₁₋₆alkyl-, or two connected substituents together with the atoms to which they are attached form a 4-6 membered lactam fused with the aryl or heteroaryl;
R³ is hydrogen, C₁₋₆alkyl, C₁₋₆alkoxy, haloC₁₋₆alkyl, halogen, amino, or —CONH—C₁₋₆alkyl-heterocyclyl;
R⁴ and R⁵ are independently hydrogen, C₁₋₆alkyl, C₃₋₇cycloalkyl, heterocyclyl-C₁₋₆alkyl, or R⁴ and R⁵ together with the N to which they are attaches form a heterocyclyl;
R⁶ is C₁₋₆alkyl or C₃₋₇cycloalkyl;
R⁷ and R⁸ are independently hydrogen or C₁₋₆alkyl.

In certain embodiments, the API is 6-(1-cyclopropylpyrazol-4-yl)-3-[difluoro-(6-fluoro-2-methylindazol-5-yl)methyl]-[1,2,4]triazolo[4,3-b]pyridazine (APL-101).

In certain embodiments, the API has a weight percentage of 10-40% in the formulation. In certain embodiments, the API has a weight percentage of 20-33% in the formulation.

In certain embodiments, the polymer is selected from the group consisting of poly(vinylpyrrolidone)-co-vinyl acetate (PVP-VA 64), hydroxypropyl methylcellulose (HPMC), poly(vinylpyrrolidone) (PVP), hydroxypropyl methylcellulose acetate succinate (HPMCAS), poly(methacrylic acid-co-methyl methacrylate) 1:1 (Eudragit L 100), hydroxypropyl methylcellulose phthalate (HPMCP-HP55) and a combination thereof.

In certain embodiments, the polymer is HPMCAS. In certain embodiments, the polymer is HPMCAS-H.

In certain embodiments, the formulation is a solid dispersion. In certain embodiments, the solid dispersion is prepared by spray drying.

In certain embodiments, the pharmaceutical composition is an oral dosage form. In certain embodiments, the dosage form is a tablet.

In another aspect, the present disclosure provides a method for treating cancer in a subject. In certain embodiments, the method comprises administering to the subject a pharmaceutical composition disclosed herein.

In certain embodiments, the cancer is selected from the group consisting of lung cancer, melanoma, renal cancer, liver cancer, myeloma, prostate cancer, breast cancer, colorectal cancer, pancreatic cancer, thyroid cancer, hematological cancer, leukemia and non-Hodgkin's lymphoma. In certain embodiments, the cancer is non-small cell lung cancer (NSCLC) or hepatocellular carcinoma.

It is contemplated that any method or composition described herein can be implemented with respect to any other method or composition described herein. Other objects, features and advantages of the present disclosure will become apparent from the following detailed description. It should be understood, however, that the detailed description and the specific examples, while indicating specific embodiments of the invention, are given by way of illustration only, since various changes and modifications within the spirit and scope of the disclosure will become apparent to those skilled in the art from this detailed description.

### BRIEF DESCRIPTION OF DRAWING

The following drawings form part of the present specification and are included to further demonstrate certain aspects of the present disclosure. The disclosure may be better understood by reference to one or more of these drawings in combination with the detailed description of specific embodiments presented herein.

FIG. 1 illustrate that an exemplary embodiment of the pharmaceutical composition comprising solid dispersion of APL-101 demonstrated increased bioavailability and exposure in a dog model.

### DETAILED DESCRIPTION OF THE INVENTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Where reference is made herein to a method comprising two or more defined steps, the defined steps can be carried out in any order or simultaneously (except where the context excludes that possibility), and the method can include one or more other steps which are carried out before any of the defined steps, between two of the defined steps, or after all the defined steps (except where the context excludes that possibility).

Where a range of value is provided, it is understood that each intervening value, to the tenth of the unit of the lower limit unless the context clearly dictate otherwise, between the upper and lower limit of that range and any other stated or intervening value in that stated range, is encompassed within the disclosure, subject to any specifically excluded limit in the stated range. Where the stated range includes one or both of the limits, ranges excluding either or both of those included limits are also included in the disclosure.

### I. Definition

It is to be understood that both the foregoing general description and the following detailed description are exemplary and explanatory only and are not restrictive of the invention as claimed. In this application, the use of the singular includes the plural unless specifically stated otherwise. In this disclosure, the term “or” is used to mean “and/or” unless explicitly indicated to refer to alternatives only or the alternatives are mutually exclusive. As used herein “another” may mean at least a second or more. Furthermore, the use of the term “including”, as well as other forms, such as “includes” and “included”, is not limiting. Also, terms such as “element” or “component” encompass both elements and components comprising one unit and elements and components that comprise more than one subunit unless specifically stated otherwise. Also, the use of the term “portion” can include part of a moiety or the entire moiety.

As used herein, the singular forms “a”, “an” and “the” include plural references unless the context clearly dictates otherwise.

The term “about” as used herein when referring to a measurable value such as an amount, a temporal duration, and the like, is meant to encompass variations of up to ±10% from the specified value. Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the disclosed subject matter. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective testing measurements.

As used herein, the term “active pharmaceutical ingredient” or “API” refers to a biologically active compound.

As used herein, the term “administering” means taking, providing or delivering a compound or composition to a desired site for biological action. These methods for administering include but are not limited to oral route, transduodenal route, parenteral injection (including intravenous, subcutaneous, intraperitoneal, intramuscular, intraarterial injection or infusion), local administration, and transrectal administration. One skilled in the art is familiar with the administration techniques that can be used for the compounds and methods as described herein, such as those discussed in Goodman and Gilman, The Pharmacological Basis of Therapeutics, current ed.; Pergamon; and Remington's, Pharmaceutical Sciences (current edition), Mack Publishing Co., Easton, Pa. In a preferred embodiment, the compounds and compositions discussed herein are administered orally.

As used herein, the term “amorphous” refers to a solid material having no long-range order in the position of its molecules. Amorphous solids are generally supercooled liquids in which the molecules are arranged in a random manner so that there is no well-defined arrangement, e.g., molecular packing, and no long-range order. Amorphous solids are generally isotropic, i.e. exhibit similar properties in all directions and do not have definite melting points. For example, an amorphous material is a solid material having no sharp characteristic crystalline peak(s) in its X-ray power diffraction (XRPD) pattern (i.e., is not crystalline as determined by XRPD). Instead, one or several broad peaks (e.g., halos) appear in its XRPD pattern. Broad peaks are characteristic of an amorphous solid.

As used herein, the term “substantially amorphous” refers to a solid material having little or no long-range order in the position of its molecules. For example, substantially amorphous materials have less than about 15% crystallinity (e.g., less than about 10% crystallinity or less than about 5% crystallinity). It is also noted that the term “substantially amorphous” includes the descriptor, “amorphous”, which refers to materials having no (0%) crystallinity.

The term “at least” followed by a number is used herein to denote the start of a range beginning with that number (which may be a range having an upper limit or no upper limit, depending on the variable being defined). For example, “at least 1” means 1 or more than 1. The term “at most” followed by a number is used herein to denote the end of a range ending with that number (which may be a range having 1 or 0 as its lower limit, or a range having no lower limit, depending upon the variable being defined). For example, “at most 4” means 4 or less than 4, and “at most 40%” means 40% or less than 40%. In this disclosure, when a range is given as “(a first number) to (a second number)” or “(a first number)-(a second number),” this means a range whose lower limit is the first number and whose upper limit is the second number. For example, 2 to 10 millimeters means a range whose lower limit is 2 millimeters, and whose upper limit is 10 millimeters.

“Cancer” as used herein refers to any medical condition characterized by malignant cell growth or neoplasm, abnormal proliferation, infiltration or metastasis, and includes both solid tumors and non-solid cancers (hematologic malignancies) such as leukemia. As used herein “solid tumor” refers to a solid mass of neoplastic and/or malignant cells. Examples of cancer or tumors include hematological malignancies, oral carcinomas (for example of the lip, tongue or pharynx), digestive organs (for example esophagus, stomach, small intestine, colon, large intestine, or rectum), peritoneum, liver and biliary passages, pancreas, respiratory system such as larynx or lung (small cell and non-small cell), bone, connective tissue, skin (e.g., melanoma), breast, reproductive organs (fallopian tube, uterus, cervix, testicles, ovary, or prostate), urinary tract (e.g., bladder or kidney), brain and endocrine glands such as the thyroid. In certain embodiments, the cancer is selected from ovarian cancer, breast cancer, head and neck cancer, renal cancer, bladder cancer, hepatocellular cancer, and colorectal cancer. In certain embodiments, the cancer is selected from a lymphoma, Hodgkin's lymphoma, non-Hodgkin's lymphoma and B-cell lymphoma.

As used herein, the term “dispersion” refers to a disperse system in which one substance, the dispersed phase, is distributed, in discrete units, throughout a second substance (the continuous phase or vehicle). The size of the dispersed phase can vary considerably (e.g. single molecules, colloidal particles of nanometer dimension, to multiple microns in size). In general, the dispersed phases can be solids, liquids, or gases. In the case of a solid dispersion, the dispersed and continuous phases are both solids. In pharmaceutical applications, a solid dispersion can include: an amorphous drug in an amorphous polymer; an amorphous drug in crystalline polymer; a crystalline drug in an amorphous polymer; or a crystalline drug in crystalline polymer. In this invention, a solid dispersion can include an amorphous drug in an amorphous polymer or an amorphous drug in crystalline polymer. In some embodiments, a solid dispersion includes the polymer constituting the dispersed phase, and the drug constitutes the continuous phase. Or, a solid dispersion includes the drug constituting the dispersed phase, and the polymer constitutes the continuous phase.

As used herein, the term “solid dispersion” generally refers to a solid dispersion of two or more components, usually one or more drugs (e.g., one drug (e.g., APL-101)) and polymer, but possibly containing other components such as surfactants or other pharmaceutical excipients, where the drug(s) (e.g., APL-101) is substantially amorphous (e.g., having about 15% or less (e.g., about 10% or less, or about 5% or less)) of crystalline drug or amorphous (i.e., having no crystalline drug), and the physical stability and/or dissolution and/or solubility of the substantially amorphous or amorphous drug is enhanced by the other components. Solid dispersions typically include a compound dispersed in an appropriate carrier medium, such as a solid state carrier. For example, a carrier comprises a polymer (e.g., a water-soluble polymer or a partially water-soluble polymer) and can include optional excipients such as functional excipients (e.g., one or more surfactants) or nonfunctional excipients (e.g., one or more fillers).

As used herein, the term “effective amount” or “therapeutically effective amount” means the amount of agent that is sufficient to prevent, treat, reduce and/or ameliorate the symptoms and/or underlying causes of any disorder or disease, or the amount of an agent sufficient to produce a desired effect on a cell. In one embodiment, a “therapeutically effective amount” is an amount sufficient to reduce or eliminate a symptom of a disease. In another embodiment, a therapeutically effective amount is an amount sufficient to overcome the disease itself.

As used herein, an “excipient” is an inactive ingredient in a pharmaceutical composition.

As used herein, the term “subject” refers to a human or any non-human animal (e.g., mouse, rat, rabbit, dog, cat, cattle, swine, sheep, horse or primate). A human includes pre and post-natal forms. In many embodiments, a subject is a human being. A subject can be a patient, which refers to a human presenting to a medical provider for diagnosis or treatment of a disease. The term “subject” is used herein interchangeably with “individual” or “patient.” A subject can be afflicted with or is susceptible to a disease or disorder but may or may not display symptoms of the disease or disorder.

The term “treatment,” “treat,” or “treating” a disease or condition as used herein include the following meanings: (i) prevention of the occurrence of the disease or condition in a subject, especially when the subject is susceptible to the disease or condition but has not been diagnosed with the disease or condition; (ii) suppression of the disease or condition, that is, inhibition of the development of the disease or condition; (iii) alleviation of the disease or condition, that is, abatement of the status of the disease or condition; or (iv) relief of the symptoms caused by the disease or condition. It is understood that treatment does not necessarily refer to a cure or complete ablation of the disease, condition, or symptoms of the disease or condition

### II. Pharmaceutical Composition

The present disclosure in one aspect provides a pharmaceutical composition comprising a formulation comprising a c-Met tyrosine kinase API (e.g., a solid dispersion of APL-101) and a polymer. As exemplified herein, the pharmaceutical composition of the present disclosure can be a powder admixture of c-Met tyrosine kinase API (e.g., a solid dispersion of APL-101) and one or more excipients described herein. Alternatively, the pharmaceutical composition can be formulated into a dosage form containing the powder admixture or a dosage form formulated to contain a compressed solid dose form of the powder admixture in addition to one or more additional functional excipients, for example, optionally a wetting agent and/or lubricant to enable the compression of the powder admixture into granules, pellets, particles, or one or more mini-tablets, the pharmaceutical composition and/or the unit dose form comprising the specified ingredients in the specified amounts. The pharmaceutical composition is capable of being formulated into a unit dose form, for example, a tablet, capsule, sachet, troches, blister pack and the like containing the powder and/or compressed form of the pharmaceutical composition of the present disclosure.

A. Active Pharmaceutical Ingredient

In certain embodiments, the API contained in the pharmaceutical composition of the present disclosure is a c-Met tyrosine kinase inhibitor. In certain embodiments, the c-Met tyrosine kinase Inhibitor is select from the compounds disclosed in U.S. Pat. No. 9,695,175 to Zhong et al., the disclosure of which is incorporated herein by reference. In certain embodiments, the API is a compound of the following formula or a pharmaceutically acceptable salt thereof or a hydrate thereof:

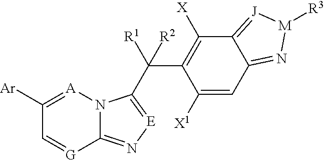

wherein:
R¹ and R² are independently hydrogen or halogen;
X and X¹ are independently hydrogen or halogen;
A and G are independently CH or N, or CH=G is replaced with a sulfur atom;
E is N;
J is CH, S or NH;
M is N or C;
Ar is aryl or heteroaryl, optionally substituted with 1-3 substituents independent selected from: C₁₋₆alkyl, C₁₋₆alkoxyl, halo C₁₋₆alkyl, halo C₁₋₆alkoxy, C₃₋₇ cycloalkyl, halogen, cyano, amino, —CONR⁴R⁵, —NHCOR⁶, —SO₂NR⁷R⁸, C₁₋₆alkoxyl-, C₁₋₆alkyl-, amino-C₁₋₆alkyl-, heterocyclyl and heterocyclyl-C₁₋₆alkyl-, or two connected substituents together with the atoms to which they are attached form a 4-6 membered lactam fused with the aryl or heteroaryl;
R³ is hydrogen, C₁₋₆alkyl, C₁₋₆alkoxy, haloC₁₋₆alkyl, halogen, amino, or —CONH—C₁₋₆alkyl-heterocyclyl;
R⁴ and R⁵ are independently hydrogen, C₁₋₆alkyl, C₃₋₇cycloalkyl, heterocyclyl-C₁₋₆alkyl, or R⁴ and R⁵ together with the N to which they are attaches form a heterocyclyl; R⁶ is C₁₋₆alkyl or C₃₋₇cycloalkyl; R⁷ and R⁸ are independently hydrogen or C₁₋₆alkyl.

In certain embodiments, the API is selected from the group consisting of

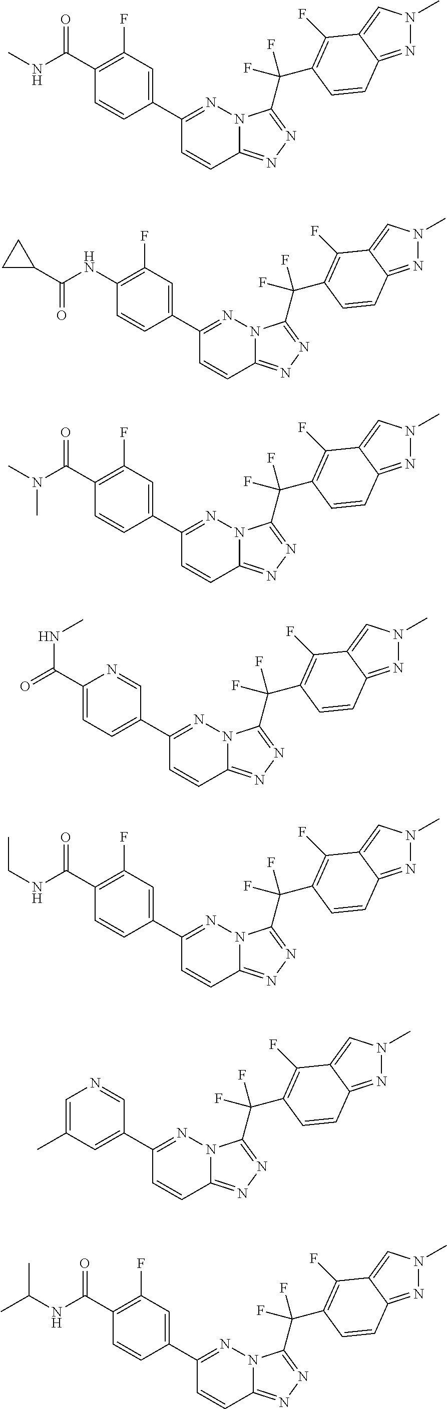

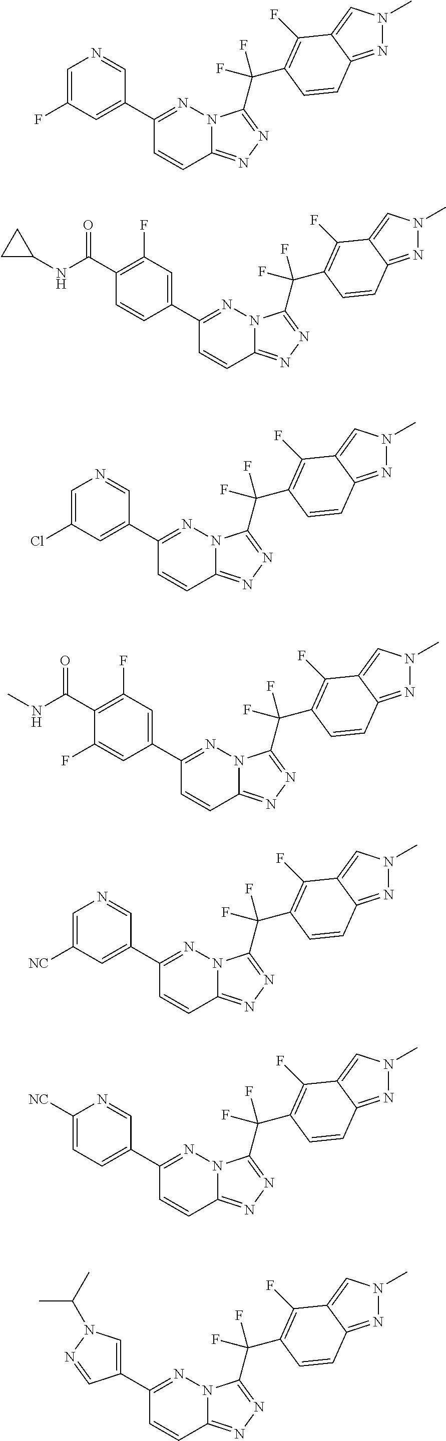

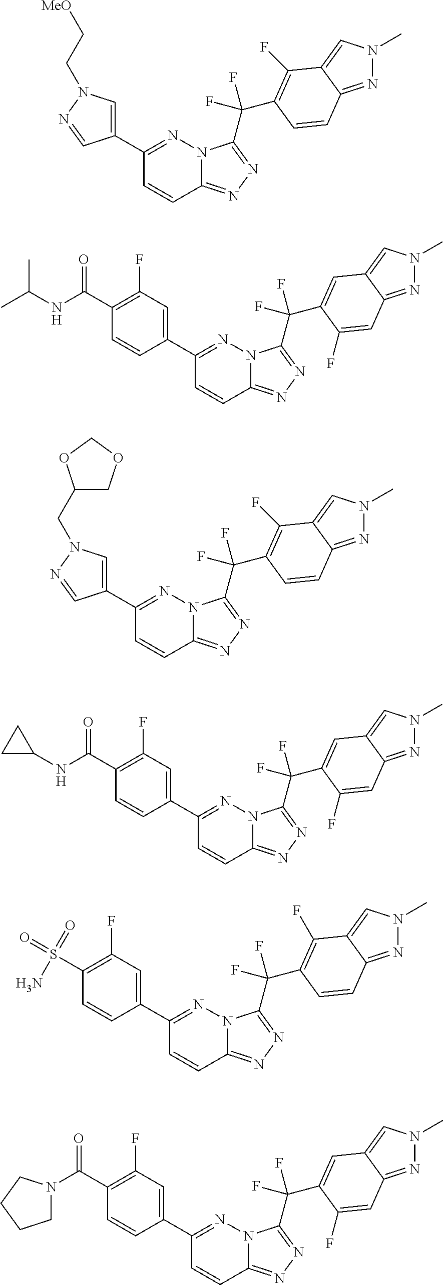

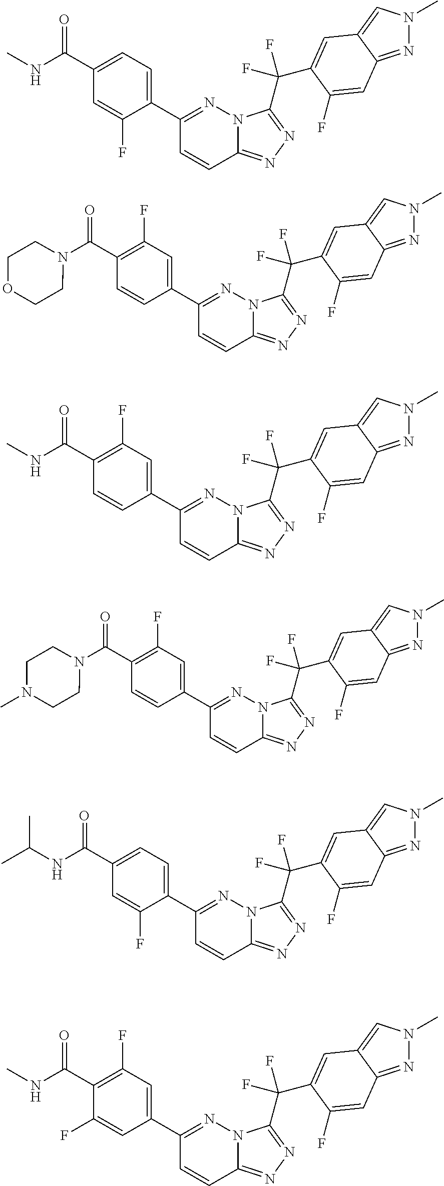

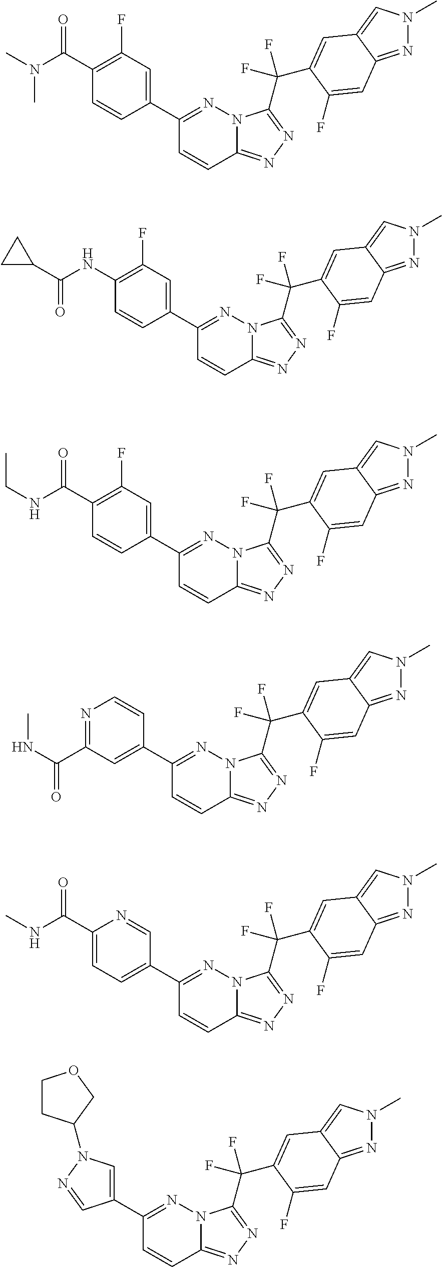

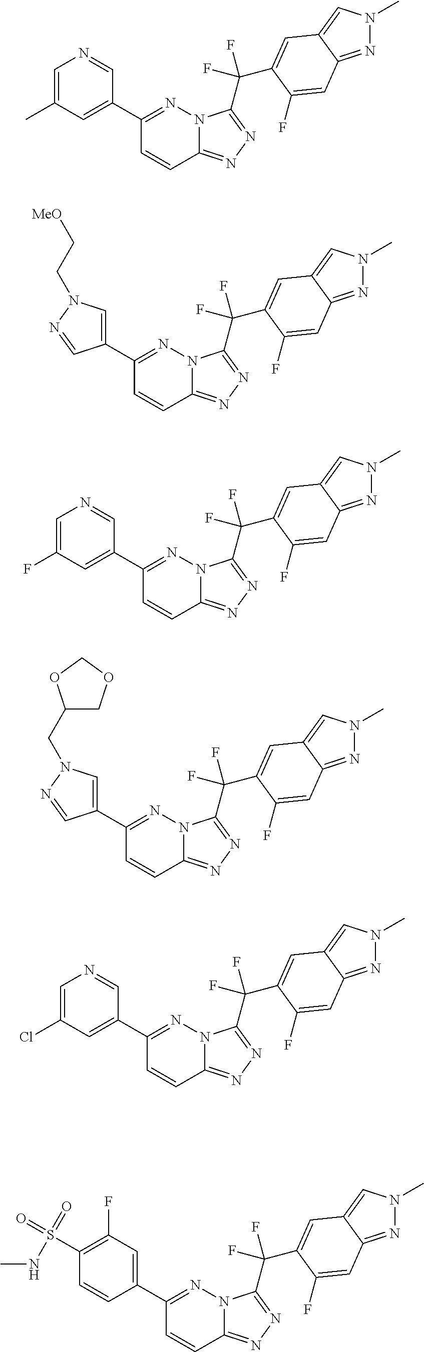

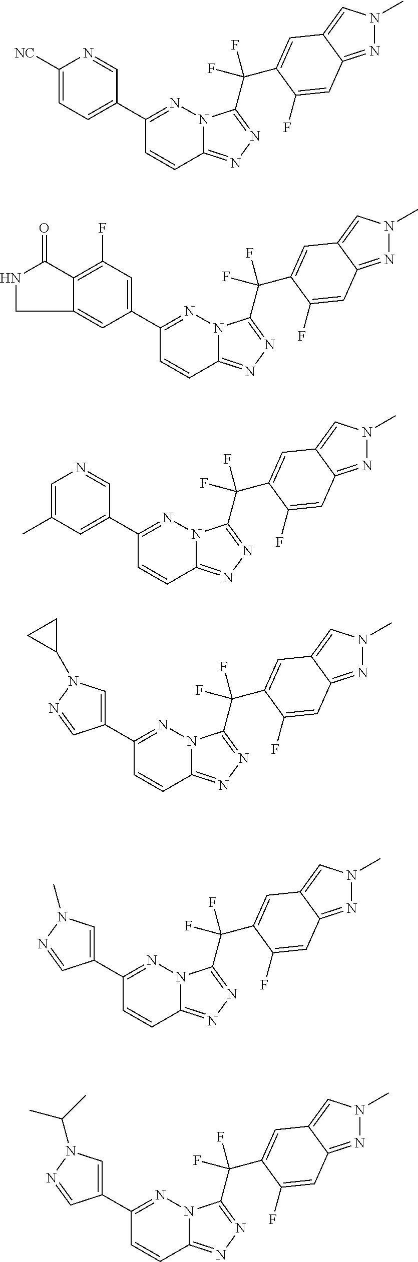

In certain embodiments, the API is 6-(1-cyclopropylpyrazol-4-yl)-3-[difluoro-(6-fluoro-2-methylindazol-5-yl)methyl]-[1,2,4]triazolo[4,3-b]pyridazine (APL-101), which has the following formula

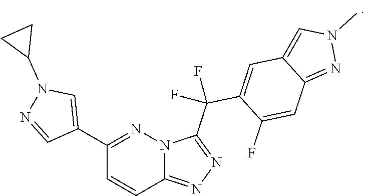 [SMILES: Cn1cc2cc(C(F)(F)c3nnc4ccc(-c5cnn(C6CC6)c5)nn34)c(F)cc2n1]

In one embodiment, the present disclosure provides a pharmaceutical composition comprising a solid dispersion of substantially amorphous API compound disclosed herein, wherein the pharmaceutical composition comprises up to about 40 wt % of substantially amorphous API compound. For instance, the pharmaceutical composition comprises about 1 wt %, 2 wt %, 3 wt %, 4 wt %, 5 wt %, about 6 wt %, about 7 wt %, about 8 wt %, about 9 wt %, about 10 wt %, about 11 wt %, about 12 wt %, about 13 wt %, about 14 wt %, about 15 wt %, about 16 wt %, about 17 wt %, about 18 wt %, about 19 wt %, about 20 wt %, about 21 wt %, about 22 wt %, about 23 wt %, about 24 wt %, about 25 wt %, about 26 wt %, about 27 wt %, about 28 wt %, about 29 wt %, about 30 wt %, about 31 wt %, about 32 wt %, about 33 wt %, about 34 wt %, about 35 wt %, about 36 wt %, about 37 wt %, about 38 wt %, about 39 wt %, about 40 wt % of substantially amorphous API compound as disclosed herein.

In certain embodiments, the pharmaceutical composition comprises about 1-40 wt %, 5-40 wt %, about 10-35 wt %, about 15-35 wt %, about 20-35 wt %, about 20-33 wt % of substantially amorphous API compound disclosed herein.

B. Excipient

In certain embodiments, the pharmaceutical composition of the present disclosure comprises a mixture of substantially amorphous API compound and an excipient.

In certain embodiments, the excipient is a polymer suitable for preparing a solid dispersion. In certain embodiments, the polymer is selected from the group consisting of poly(vinylpyrrolidone)-co-vinyl acetate (PVP-VA 64), hydroxypropyl methylcellulose (HPMC), poly(vinylpyrrolidone) (PVP), hydroxypropyl methylcellulose acetate succinate (HPMCAS), poly(methacrylic acid-co-methyl methacrylate) 1:1 (Eudragit L 100), hydroxypropyl methylcellulose phthalate (HPMCP-HP55) and a combination thereof. In preferred embodiments, the polymer is HPMCAS. In more preferred embodiments, the polymer is HPMCAS-H.

In certain embodiments, the pharmaceutical composition comprises a solid dispersion comprising about 40-95 wt % of the polymer disclosed herein. For instance, the pharmaceutical composition comprises about 40 wt %, 41 wt %, 42 wt %, 43 wt %, 44 wt %, 45 wt %, 46 wt %, 47 wt %, 48 wt %, 49 wt %, 50 wt %, 51 wt %, 52 wt %, 53 wt %, 54 wt %, 55 wt %, 56 wt %, 57 wt %, 58 wt %, 59 wt %, 60 wt %, about 61 wt %, about 62 wt %, about 63 wt %, about 64 wt %, about 65 wt %, about 66 wt %, about 67 wt %, about 68 wt %, about 69 wt %, about 70 wt %, about 71 wt %, about 72 wt %, about 73 wt %, about 74 wt %, about 75 wt %, about 76 wt %, about 77 wt %, about 78 wt %, about 79 wt %, about 80 wt %, about 81 wt %, about 82 wt %, about 83 wt %, about 84 wt %, about 85 wt %, about 86 wt %, about 87 wt %, about 88 wt %, about 89 wt %, about 90 wt %, about 91 wt %, about 92 wt %, about 93 wt %, about 94 wt %, about 95 wt % of the polymer as disclosed herein.

In certain embodiments, the pharmaceutical composition comprises about 40%-95 wt %, 45-95 wt %, 50-95 wt %, 55-90 wt %, 60-95 wt %, about 65-90 wt %, about 65-85 wt %, about 65-80 wt %, about 66-80 wt % of the polymer disclosed herein.

In certain embodiment, the pharmaceutical composition of the present disclosure further comprises a second excipient, for example, optionally a wetting agent and/or lubricant to enable the compression of the pharmaceutical composition into granules, pellets, particles, or one or more mini-tablets, and/or the unit dose form comprising the specified ingredients in the specified amounts. The suitable second excipients are compatible with the ingredients of the pharmaceutical composition disclosed herein, i.e., they do not substantially reduce the solubility, the chemical stability, the physical stability, or the biological activity of the pharmaceutical composition. Example of the second excipient contained in the pharmaceutical composition include, without limitation, fillers, sweeteners, disintegrants, wetting agents, glidants, lubricants, colorants, flavoring agent or combinations thereof. It is noted that some of the second excipients may service more than one function, such as some fillers can also be sweeteners and some disintegrants can also be wetting agents (e.g. mannitol is filler and sweetener, SLS is a wetting agent and lubricant).

Examples of suitable filler can include, but are not limited to, mannitol, lactose, sucrose, dextrose, maltodextrin, sorbitol, xylitol, powdered cellulose, polyhydric alcohols, microcrystalline cellulose, silicified microcrystalline cellulose, cellulose acetate, methylcellulose, ethylcellulose, hydroxyethylcellulose, methylhydroxyethylcellulose, talc, starch (i.e. potato starch), pregelatinized starch, dibasic calcium phosphate, calcium sulfate and calcium carbonate.

Examples of suitable sweeteners include, but are not limited to, monosaccharides, disaccharides and polysaccharides. Examples of suitable sweeteners include both natural and artificial sweeteners. Examples can include, but are not limited to, glucose, sucrose, maltose, mannose, dextrose, fructose, lactose, trehalose, maltitol, lactitol, xylitol, sorbitol, mannitol, tagatose, glycerin, erythritol, isomalt, maltose, sucralose, aspartame, neotame, alitame, neohesperidin dihydrochalcone, cyclamate (i.e. sodium cyclamate), thaumatin, acesulfame potassium, saccharin, and saccharin sodium.

Disintegrants suitable for the present disclosure enhance the dispersal of the pharmaceutical composition. Exemplary disintegrants include: croscarmellose sodium (e.g., AcDiSol), sodium alginate, calcium alginate, alginic acid, starch, pregelatinized starch, sodium starch glycolate, polyvinylpyrrolidone, co polymers of polyvinylpyrrolidone, crospovidone, carboxymethylcellulose calcium, cellulose and its derivatives, carboxymethylcellulose sodium, soy polysaccharide, clays, gums (i.e. guar gum), an ion exchange resin, an effervescent system based on food acids and an alkaline carbonate component, and sodium bicarbonate.

Wetting agents and/or surfactants suitable for the present invention can enhance the solubility or the wettability of the pharmaceutical composition. In some embodiments, the one or more wetting agents include one or more surfactants. Examples of wetting agents/surfactants may include, but are not limited to the following: sodium lauryl sulfate (also called sodium dodecyl sulfate (SDS)), cetostearyl alcohol, cetomacrogol emulsifying wax, gelatin, casein, docusate sodium, benzalkonium chloride, calcium stearate, polyethylene glycols, phosphates, polyoxyethylene sorbitan fatty acid esters (e.g. Polysorbate 80, Polysorbate 20), gum acacia, cholesterol, tragacanth, polyoxyethylene 20 stearyl ethers, polyoxyethylene alkyl ethers, polyoxyethylene castor oil derivatives, pegylated hydrogenated castor oils, sorbitan esters of fatty acids, Vitamin E or tocopherol derivatives, vitamin E TPGS, tocopheryl esters, lecithin, phospholipids and their derivatives, poloxamers, stearic acid, oleic acid, oleic alcohol, cetyl alcohol, mono and diglycerides, propylene glycol esters of fatty acids, glycerol esters of fatty acids (i.e. glycerol monostearate), ethylene glycol palmitostearate, polyoxylglycerides, propylene glycol monocaprylate, propylene glycol monolaurate, alkyl aryl polyether alcohols (Triton®) and polyglyceryl oleate.

A “glidant” is a substance to promote powder flow by reducing interparticle friction and cohesion. Examples of the glidants may include, but are not limited to, talc, colloidal silica (e.g., Cabosil M-5P), precipitated silica, magnesium oxide, magnesium silicate, leucine and starch.

Lubricants suitable for the present invention improve the compression and ejection of compressed pharmaceutical compositions from a die. Lubricants may further have anti-sticking or anti-tacking properties, and minimize sticking in various operations of the present disclosure, including operations such as encapsulation. Examples of the lubricants may include, but are not limited to, talc, fatty acid, stearic acid, magnesium stearate, calcium stearate, sodium stearate, stearic acid, glyceryl monostearate, sodium lauryl sulfate, sodium stearyl fumarate, hydrogenated oils (i.e. hydrogenated vegetable oil), polyethylene glycol, fatty alcohol, fatty acid ester, glyceryl behenate, mineral oil, vegetable oil, leucine, sodium benzoate, or a combination thereof.

In certain embodiments, the second excipient contained in pharmaceutical composition is about 1 wt %, 2 wt %, 3 wt %, 4 wt %, 5 wt %, 6 wt %, 7 wt %, 8 wt %, 9 wt %, 10 wt %, 11 wt %, 12 wt %, 13 wt %, 14 wt %, 15 wt %, 16 wt %, 17 wt %, 18 wt %, 19 wt %, 20 wt % 25 wt % 30 wt %, 35 wt %, 40 wt %, 45 wt % 50 wt % 55%, 60%, 65%, 70%, 75%, 80%, 85%, 90%, 95% of the pharmaceutical composition.

Pharmaceutical compositions of the present disclosure can optionally comprise one or more colorants, flavors, and/or fragrances to enhance the visual appeal, taste, and/or scent of the composition. Suitable colorants, flavors, or fragrances are compatible with the ingredients of the pharmaceutical composition, i.e., they do not substantially reduce the solubility, the chemical stability, the physical stability or the biological activity of the pharmaceutical composition.

Suitable flavoring agents can include, for example, flavors, which are known to those of skill in the art, such as, for example, natural flavors, artificial flavors, and combinations thereof. Flavoring agents may be chosen, e.g., from synthetic flavor oils and flavoring aromatics and/or oils, oleoresins, extracts derived from plants, leaves, flowers, fruits, and the like, and combinations thereof. Non-limiting examples of flavor oils include spearmint oil, cinnamon oil, oil of wintergreen (methyl salicylate), peppermint oil, clove oil, bay oil, anise oil, eucalyptus oil, thyme oil, cedar leaf oil, oil of nutmeg, allspice, oil of sage, mace, oil of bitter almonds, and cassia oil. Suitable flavoring agents also include, for example, artificial, natural and synthetic flower derived or fruit flavors such as vanilla, ethyl vanillin, citrus oils (e.g., lemon, orange, tangerine, lime, and grapefruit), and fruit essences (e.g., natural and/or artificial flavor of apple, pear, peach, orange, grape, strawberry, raspberry, cherry, plum, pineapple, and apricot), and the like, and combinations thereof. The flavoring agents may be used in liquid or solid form and, as indicated above, may be used individually or in admixture. Other flavoring agents can include, for example, certain aldehydes and esters, e.g., cinnamyl acetate, cinnamaldehyde, citral diethylacetal, dihydrocarvyl acetate, eugenyl formate, p-methylamisol, and the like, and combinations thereof.

C. Solid Dispersion Formulation

In certain embodiments, the pharmaceutical composition disclosed herein comprises a formulation which is a solid dispersion, wherein the solid dispersion comprises a mixture of substantially amorphous API and a polymer suitable for preparing the solid dispersion.

In certain embodiments, the solid dispersion comprises up to about 40 wt % of substantially amorphous API compound. For instance, the solid dispersion comprises about 5 wt %, about 6 wt %, about 7 wt %, about 8 wt %, about 9 wt %, about 10 wt %, about 11 wt %, about 12 wt %, about 13 wt %, about 14 wt %, about 15 wt %, about 16 wt %, about 17 wt %, about 18 wt %, about 19 wt %, about 20 wt %, about 21 wt %, about 22 wt %, about 23 wt %, about 24 wt %, about 25 wt %, about 26 wt %, about 27 wt %, about 28 wt %, about 29 wt %, about 30 wt %, about 31 wt %, about 32 wt %, about 33 wt %, about 34 wt %, about 35 wt %, about 36 wt %, about 37 wt %, about 38 wt %, about 39 wt %, about 40 wt % of substantially amorphous API compound as disclosed herein. In certain embodiments, the solid dispersion comprises about 5-40 wt %, about 10-35 wt %, about 15-35 wt %, about 20-35 wt %, about 20-33 wt % of substantially amorphous API compound disclosed herein.

The polymer suitable for preparing the solid dispersion has been disclosed supra. In certain embodiments, the solid dispersion comprises about 60-95 wt % of the polymer. For instance, the solid dispersion comprises about 60 wt %, about 61 wt %, about 62 wt %, about 63 wt %, about 64 wt %, about 65 wt %, about 66 wt %, about 67 wt %, about 68 wt %, about 69 wt %, about 70 wt %, about 71 wt %, about 72 wt %, about 73 wt %, about 74 wt %, about 75 wt %, about 76 wt %, about 77 wt %, about 78 wt %, about 79 wt %, about 80 wt %, about 81 wt %, about 82 wt %, about 83 wt %, about 84 wt %, about 85 wt %, about 86 wt %, about 87 wt %, about 88 wt %, about 89 wt %, about 90 wt %, about 91 wt %, about 92 wt %, about 93 wt %, about 94 wt %, about 95 wt % of the polymer as disclosed herein.

In certain embodiments, the solid dispersion optionally comprises a second excipient that is suitable for preparing a solid dispersion.

In certain embodiment, the pharmaceutical composition comprises 30-100 wt % of the solid dispersion. For instance, the pharmaceutical composition comprises about 30 wt %, about 35 wt %, about 40 wt %, about 45 wt %, about 50 wt %, about 55 wt %, about 60 wt %, about 65 wt %, about 70 wt %, about 75 wt %, about 80 wt %, about 85 wt %, about 90 wt %, about 95 wt %, about 100 wt % of the solid dispersion.

It can be understood that the weight percentage of the API or polymer can be calculated based on the percentage of the API or polymer in the formulation, e.g., solid dispersion, and the percentage of the formulation in the pharmaceutical composition. For example, in a pharmaceutical composition comprising 80 wt % of solid dispersion, which contains 40 wt % API. The weight percentage of API in the pharmaceutical composition is 32%.

D. Dosage Form

In certain embodiments, the pharmaceutical composition disclosed herein is formulated into a solid dosage form or unit dosage form, such as a granule, pellet, tablet and the like. In certain embodiments, the solid dosage form or unit dosage form comprises the solid dispersion disclosed supra with the addition of one or more functional excipients, for example, a disintegrant, glidant, lubricant, filler and/or a wetting agent to facilitate compression of the pharmaceutical composition, and to facilitate disintegration and dissolution of the pharmaceutical composition. The solid dosage form such as granule, pellet, particle, tablet and the like can be formulated into unit dosage forms such as capsules, pouches, packets, sachets, bottles and blister packs containing one or more such solid dosage forms. The number of solid dosage forms required for each unit dosage forms will depend on the concentration of API in each solid dosage form, e.g., in each granule, pellet), and the required final amount of API required for the unit dosage form.

In certain embodiments, the solid dosage form disclosed herein is a tablet. In certain embodiments, the tablet is about 2 mm, 3 mm, 4 mm, 5 mm, 6 mm, 7 mm, 8 mm, 9 mm or 10 mm in size.

In certain embodiments, the solid dosage form contains about 5-50 mg API (e.g., APL-101), for example, about 5 mg, 10 mg, 15 mg, 20 mg, 25 mg, 30 mg, 35 mg, 40 mg, 45 mg, or 50 mg API.

### III. Methods of Producing a Pharmaceutical Composition

Another aspect of the present disclosure provides a method of producing a pharmaceutical composition disclosed herein. In certain embodiments, the method comprises providing an admixture of a solid dispersion of substantially amorphous or amorphous API compound and a polymer, one or more fillers, a sweetener, a disintegrant, optionally a wetting agent, a glidant; and a lubricant, and compressing the admixture into a tablet. In some embodiments, the tablet has an increased dissolution and bioavailability.

Each of the ingredients of this admixture is described above and in the Examples below. Furthermore, the admixture can comprise optional additives such as one or more colorants, one or more flavors, and/or one or more fragrances as described above. The relative concentrations (e.g., wt %) of each of these ingredients (and any optional additives) in the admixture is also presented above and in the Examples below. The ingredients constituting the admixture can be provided sequentially or in any combination of additions; and, the ingredients or combination of ingredients can be provided in any order.

The method of producing solid dispersion is known in the art and has been describer earlier (see, e.g., Tran P et al., Pharmaceutics 11(3): 132 (2019)). In general, solid dispersion can be prepared by several methods including solvent evaporation, melting and supercritical fluid technology.

Spray-drying is one of the oldest methods for drying materials, especially thermally-sensitive materials such as pharmaceuticals. In this method, the drug is dissolved in a suitable solvent, and the carrier is dissolved in water to prepare the feed solution. Then, the two solutions are mixed by sonication or other suitable methods until the solution is clear. In the procedure, the feed solutions were firstly sprayed in a drying chamber via a high-pressure nozzle to form fine droplets. The formed droplets are composed of drying fluid (hot gas) and form particles of nano or micro size. Clinically, the spray-drying method has been widely used for preparation of solid dispersion for improving solubility and bioavailability of poorly water-soluble drugs such as nilotinib, spironolactone, valsartan, rebamipide, and artemether.

In certain embodiments, the method of producing a pharmaceutical composition comprises providing a solid dispersion of substantially amorphous APL-101 and a polymer disclosed herein, such as HPMCAS-H; mixing the solid dispersion with one or more additional excipients, such as a filler, a sweetener, a disintegrant, awetting agent, a glidant and a lubricant until the admixture is substantially homogenous; and compressing the admixture into a solid dosage form as described above or in the Examples below. For example, the admixture is mixed by stirring, blending, shaking, or the like using hand mixing, a mixer, a blender, any combination thereof; or the like. When ingredients or combinations of ingredients are added sequentially, mixing can occur between successive additions, continuously throughout the ingredient addition, after the addition of all of the ingredients or combinations of ingredients, or any combination thereof. In addition, prior to or subsequent to each mixing step, the blended ingredients can be further sieved by passing the ingredients or blend through an appropriately sized mesh screen or delumped using a mill with an appropriate screen size. The admixture is mixed until it has a substantially homogenous composition. The admixture/powder blend can be further filled in an appropriate dosage form or package, i.e. it can be encapsulated or filled into pouches, packets, sachets, bottles, etc. for administration. The powder blend can be further processed into granules or pellets or tablet and the like.

### IV. Method of Treatment

In one aspect, the present disclosure provides methods of using pharmaceutical compositions as disclosed herein to treat diseases, including without limitation, cancers.

A. Cancers

While hyperproliferative diseases can be associated with any disease which causes a cell to begin to reproduce uncontrollably, the prototypical example is cancer. One of the key elements of cancer is that the cell's normal apoptotic cycle is interrupted and thus agents that interrupt the growth of the cells are important as therapeutic agents for treating these diseases.

Examples of cancer can be generally categorized into solid tumors and hematologic malignancies. Solid tumors include but are not limited to, non-small cell lung cancer (squamous/non-squamous), small cell lung cancer, renal cell cancer, colorectal cancer, colon cancer, ovarian cancer, breast cancer (including basal breast carcinoma, ductal carcinoma and lobular breast carcinoma), pancreatic cancer, gastric carcinoma, bladder cancer, esophageal cancer, mesothelioma, melanoma, head and neck cancer, thyroid cancer, sarcoma, prostate cancer, glioblastoma, cervical cancer, thymic carcinoma, melanoma, myelomas, mycoses fungoids, merkel cell cancer, hepatocellular carcinoma (HCC), fibrosarcoma, myxosarcoma, liposarcoma, chondrosarcoma, osteogenic sarcoma, and other sarcomas, synovioma, mesothelioma, Ewing's tumor, leiomyosarcoma, rhabdomyosarcoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, hepatoma, bile duct carcinoma, choriocarcinoma, Wilms' tumor, cervical cancer, testicular tumor, seminoma, polycythemia vera, mast cell derived tumors, EBV-positive and -negative PTLD, nasopharyngeal carcinoma, spinal axis tumor, brain stem glioma, astrocytoma, medulloblastoma, craniopharyogioma, ependymoma, pinealoma, hemangioblastoma, acoustic neuroma, oligodendroglioma, menangioma, melanoma, neuroblastoma and retinoblastoma.

Hematologic malignancies include but are not limited to acute lymphocytic leukemia, acute myeloid leukemia (AML), B-cell leukemia, blastic plasmacytoid dendritic cell neoplasm (BPDCN), chronic lymphoblastic leukemia (CLL), chronic lymphocytic leukemia, chronic myeloid leukemia (CML), chronic myelomonocytic leukemia (CMML), chronic myelomonocytic leukemia (CMML), classical Hodgkin lymphoma (CHL), diffuse large B-cell lymphoma (DLBCL), extranodal NK/T-cell lymphoma, hairy cell leukemia, heavy chain disease, HHV8-associated primary effusion lymphoma, lymphoid malignancy, multiple myeloma (MM), myelodysplasia, myelodysplastic syndrome (MDS), non-Hodgkin's lymphoma, plasmablastic lymphoma, pre-B acute lymphocytic leukemia (Pre-B ALL), primary CNS lymphoma, primary mediastinal large B-cell lymphoma, T-cell/histiocyte-rich B-cell lymphoma, and Waldenstrom's macroglobulinemia.

B. Administration of the Pharmaceutical Composition

In some embodiments, the present disclosure provides methods of treating a disease in a subject, comprising administering to the subject a therapeutically effective amount of the API (e.g., APL-101) contained in the pharmaceutical composition provided herein.

The therapeutically effective amount (when used alone or in combination with other agents such as chemotherapeutic agents) of the API contained in a pharmaceutical composition provided herein will depend on various factors known in the art, such as for example type of disease to be treated, body weight, age, past medical history, present medications, state of health of the subject, immune condition and potential for cross-reaction, allergies, sensitivities and adverse side-effects, as well as the administration route and the type, the severity and development of the disease and the discretion of the attending physician or veterinarian. In certain embodiments, the pharmaceutical composition provided herein may be administered at a therapeutically effective dosage of the API of about 0.001 mg/kg to about 100 mg/kg one or more times per day or per week (e.g., about 0.001 mg/kg, about 0.3 mg/kg, about 0.5 mg/kg, about 1 mg/kg, about 3 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, or about 100 mg/kg one or more times per day or per week). In certain embodiments, the pharmaceutical composition is administered at a dosage of the API of about 50 mg/kg or less, and in certain embodiments the dosage is 20 mg/kg or less, 10 mg/kg or less, 3 mg/kg or less, 1 mg/kg or less, 0.3 mg/kg or less, 0.1 mg/kg or less, or 0.01 mg/kg or less, or 0.001 mg/kg or less. In certain embodiments, the administration dosage may change over the course of treatment. For example, in certain embodiments the initial administration dosage may be higher than the subsequent administration dosages. In certain embodiments, the administration dosage may vary over the course of treatment depending on the reaction of the subject.

Dosage regimens may be adjusted to provide the optimum desired response (e.g., a therapeutic response). In certain embodiments, the pharmaceutical composition provided herein is administered to the subject at one time or over a series of treatments. In certain embodiments, the pharmaceutical composition provided herein is administered to the subject by one or more separate administrations depending on the type and severity of the disease.

In some embodiments, the pharmaceutical composition provided herein can be administered alone or in combination with one or more additional therapeutic agents or means. For example, the pharmaceutical composition provided herein may be administered in combination with a second therapy, such as radiation therapy, chemotherapy, targeted therapies, gene therapy, immunotherapy, hormonal therapy, angiogenesis inhibition, palliative care, surgery for the treatment of cancer (e.g., tumorectomy), one or more anti-emetics or other treatments for complications arising from chemotherapy, or a second therapeutic agent for use in the treatment of cancer or any medical disorder, for example, another antibody, therapeutic polynucleotide, chemotherapeutic agent(s), anti-angiogenic agent, cytokines, other cytotoxic agent(s), growth inhibitory agent(s). In certain of these embodiments, the pharmaceutical composition provided herein may be administered simultaneously with the one or more additional therapeutic agents, and in certain of these embodiments the pharmaceutical composition and the additional therapeutic agent(s) may be administered as part of the same pharmaceutical composition. However, a pharmaceutical composition administered “in combination” with another therapeutic agent does not have to be administered simultaneously with or in the same composition as the agent. A pharmaceutical composition administered prior to or after another agent is considered to be administered “in combination” with that agent as the phrase is used herein, even if the pharmaceutical composition and second agent are administered via different routes. Where possible, additional therapeutic agents administered in combination with the pharmaceutical composition provided herein are administered according to the schedule listed in the product information sheet of the additional therapeutic agent, or according to the Physicians' Desk Reference 2003 (Physicians' Desk Reference, 57th Ed; Medical Economics Company; ISBN: 1563634457; 57th edition (November 2002)) or protocols well known in the art.

The following examples are provided to better illustrate the claimed invention and are not to be interpreted as limiting the scope of the invention. All specific compositions, materials, and methods described below, in whole or in part, fall within the scope of the present invention. These specific compositions, materials, and methods are not intended to limit the invention, but merely to illustrate specific embodiments falling within the scope of the invention. One skilled in the art may develop equivalent compositions, materials, and methods without the exercise of inventive capacity and without departing from the scope of the invention. It will be understood that many variations can be made in the procedures herein described while still remaining within the bounds of the present invention. It is the intention of the inventors that such variations are included within the scope of the invention.

### Example 1

Materials and Methods

Excipients and Equipment

Potential excipients for spray dried solid dispersions and tablet development and manufacturing were of compendial or USP grade and selected based on prior experience at Patheon Development Services Inc (Bend, OR). A full list of excipients and equipment utilized for this body of work can be found in Table 1. Unless indicated otherwise, percent compositions of solutions or solid dispersions are described on a weight:weight basis.

TABLE 1
Materials and Equipment

| Material and | Trade Name | Abbreviation or | |
|---|---|---|---|
| Equipment | or Model | Equipment ID | Manufacturer |
| Acetone | Acetone | Acetone | Fisher |
| Methanol | Methanol | MeOH | Fisher |
| Ethanol | Ethanol | EtOH | Sigma-Aldrich |
| Dichloromethane | Methylene | DCM | Fisher |
| | Chloride | | |
| Povidone | Kollidon 30 | PVP K30 | BASF |
| Polyvinylpyrrolidone/ | Kollidon | PVP-VA | BASF |
| Vinyl acetate | VA 64 | | |
| copolymer | | | |
| Hypromellose | Methocel E3 | HPMC-E3LV | BASF |
| | Premium LV | | |
| Hypromellose acetate | AQOAT-MG | HPMCAS-M | Shin Etsu |
| succinate MG grade | | | |
| Hypromellose acetate | AQOAT-HG | HPMCAS-H | Shin Etsu |
| succinate HG grade | | | |
| Spray Dryer | B-290 | RD-038 | Buchi |
| Condenser | B-295 | RD-039 | Buchi |
| Tray Dryer | 12083308 | RD-044 | VWR |

Differential Scanning Calorimetry (DSC)

DSC was performed using a TA Instruments Discovery DSC2500 differential scanning calorimeter equipped with a TA instruments Refrigerated Cooling System 90 operating in either modulated or ramp mode. DSC was used to measure thermodynamic events and characteristics of APL-101 bulk API and subsequent Spray Dried Intermediates (SDIs). Events observed include the glass transition temperature (Tg) defined as the temperature at which amorphous materials transition from a low mobility glassy state to a high mobility rubbery state, cold crystallization (Tc), defined as a crystallization event at a temperature lower than the melt temperature, and melting temperature (Tm). SDI samples were placed in non-hermetic aluminum pans and heated at a constant rate of 2.0° C./min over a 25-200° C. temperature range. The system was purged by nitrogen flow at 50 mL/min to ensure inert atmosphere through the course of measurement. APL-101 was initially analyzed by standard DSC with a heating rate of 10° C./min ramping up to 221° C. Amorphous API was successfully created by rapidly quenching liquefied APL-101 using the RCS. The resulting amorphous API was analyzed by modulated DSC to determine the glass transition temperature. The API Tm/Tg ratio is a strong indicator of a molecule's crystal lattice energy and its propensity to crystallize, providing an indicator of formulation design space where ASD will be stable at a certain drug:polymer ratio.

X-Ray Powder Diffraction (XRPD)

XRPD was performed using a Rigaku Miniflex 6G X-ray diffractometer to evaluate the crystallinity of bulk API and subsequent spray dried materials. Amorphous materials give an “amorphous halo” diffraction pattern, absent of discrete peaks that would be found in a crystalline material. Samples were irradiated with monochromatized Cu Kα radiation and analyzed between 5° and 40° with a continuous scanning mode. Samples were rotated during analysis to minimize preferred orientation effects.

Particle Morphology by Scanning Electron Microscope (SEM)

SEM samples were prepared by dispersing powder onto an adhesive carbon-coated sample stub and coating with a thin conductive layer of gold-palladium using a Cressington 108 Auto. Samples were analyzed using a FEI Quanta 200 SEM fitted with an Everhart-Thornley (secondary electron) detector operating in high vacuum mode. Micrographs at various magnifications were captured for qualitative particle morphology analysis.

Particle Size Distribution (PSD) by Light Diffraction

The particle size distribution of SDI samples was determined by laser diffraction using a Mastersizer 3000 with an Aero S unit (Malvern Instruments). About 100 mg samples were added to the standard venturi disperser with a hopper gap of 1.0 mm and then fed into the dispersion system. The feed rate of 15-25% was adjusted to keep the laser obscuration level at 0.1-20%. Compressed air at 1.5 bar was used to transport and suspend the sample particles through the optical cell. A measurement time of 3 seconds was used, and background measurements were made using air for 10 seconds. Dv10, Dv50 and Dv90 diameters were used to characterize the particle size distribution of powders. For instance, the Dv50 diameter is the diameter at which 50% of a sample's volume is comprised of smaller particles.

Water Content by Coulometric Karl Fisher (KF) Titration

Samples were analyzed for water content by a Metrohm 831 Karl Fischer Coulometric Titrator with a Metrohm 874 oven processor. About 100 mg samples were sealed in 6 mL crimp vials followed by measurement of water content with the following parameters: Reagent Hydranal Coulomat AG-Oven, Oven temperature 130° C. and sample extraction time 300 seconds.

Assay and Impurities Analysis by HPLC

Assay and impurities of SDI samples were evaluated using an experimental HPLC method. The HPLC method utilized was based on the API manufacturing HPLC method for APL-101. The method demonstrated passing system suitability criteria for early development work, including but not limited to resolution, standard agreement, tailing, and signal to noise. Blank interference was observed.

Residual Solvent by Gas Chromatography Headspace Sampling

The residual solvent content of SDIs was measured by GC-HS after secondary drying. Measurements were made using an HP 6890 series GC equipped with an Agilent 7697A headspace sampler. A 30 m×0.32 mm×1.8μ capillary column with 6% cyanopropylphenyl 94% dimethylpolysiloxane GC column was used for the testing. GC samples were prepared by dissolving ˜100 mg sample in 4 mL dimethyl sulfoxide (DMSO).

Biorelevant Dissolution Performance

Solvent shift dissolution experiments were performed by dissolving bulk APL-101 in DMSO at 50 mg/mL, rendering it amorphous. Each polymer was dissolved in FaSSIF (pH 6.8), which was prepared per the manufacturer's directions. 100 μL of the API in DMSO stock solution was introduced into 10 mL of polymer/FaSSIF solution while stirring with a magnetic stir bar at 800 rpm (dilution of APL-101 to 1 mg/mL). The purpose of the solvent shift experiment is to rank order polymers for formulation screening, based on their ability to maintain supersaturation of the amorphous API as it is dosed into FaSSIF. 0.5 mL aliquots were taken at the following time points: 5, 15, 30, 45, and 60 minutes. Aliquots were transferred to a 1.5 mL centrifuge tube and spun down at 14,000 rpm for 3 min. 100 μL of supernatant was sampled and diluted with 900 μL 3:1 ACN:water for HPLC analysis.

Biorelevant drug dissolution performance for bulk API and subsequent ASDs was evaluated by Patheon's two stage ‘gastric transfer’ non-sink dissolution test, which simulates pH and bile salt concentrations for both gastric and intestinal exposure in a simple to perform assay. Pre-weighed powder is briefly suspended in media (e.g. by 10 sec vortex mixing with 4.0 mL media) and transferred to a pre-heated (37° C.) volume of 50 mL of 0.1N HCl (aq) pH ˜1.0, without pepsin or bile salts), in a USP Type 2 mini-vessel (100 mL total vessel volume) while stirring (paddles) at 100 rpm. After 30 minutes of gastric pH exposure, an equal volume of PBS buffered, 2× concentrated fasted-state simulated intestinal fluid (FaSSIF) is added to the HCl, resulting in a final pH of 6.8 in FaSSIF (100 mM PBS containing 2.24 mg/mL FaSSIF/FeSSIF/FaSSGF powder (Biorelevant Inc.) in a total volume of 100 mL. Aliquots (1.0 mL) of dissolution media are taken at the following time points: 10, 25, 35, 50, 70, 120 and 210 minutes. The first two aliquots are taken from the simulated gastric media, with the remaining five aliquots from the intestinal media. Aliquots are spun-down (13000 rpm) to pellet out undissolved solids, and the supernatant sampled and further diluted in an appropriate diluent to determine API total drug concentration (e.g. free and colloidal/polymer-bound drug in solution) utilizing a suitable HPLC method. The volume of FaSSIF added is adjusted to account for the sampling volume removed prior to gastric transfer (typically 4×1.0 mL). Initial API concentration in dissolution samples was determined utilizing a HPLC method.

### Example 2

This example illustrates the analysis and property assessment of APL-101.

Thermal properties of bulk APL-101 were measured by DSC. Three API lots were tested, and it was determined that there were two API forms, Forms A and B, both of which are thought to be hydrates. Fast ramping experiments were performed on three lots of API, during which a sharp endothermic melting event (Tm) was observed at 202° C. for Form A and 223° C. for Form B. The Tg was measured via a melt-quench technique, heating past its melting temperature and rapidly cooling to trap the molten material in an amorphous state. The resulting sample was analyzed by MDSC and a Tg of 89° C. was observed in Form B with no crystallization up to 250° C. This results in a Tm/Tg ratio of 1.37 indicative of moderate physical stability.

Diffraction patterns of all three lots of bulk APL-101 were collected using XRPD. The diffraction patterns indicate a crystalline material, consistent with thermal analysis.

Surface morphology of the bulk API particles was characterized using scanning electron microscopy.

Organic solubility of Form B APL-101 was determined visually in common spray drying solvents. DCM:MeOH 80:20 was selected as the spray drying solvent based on sufficient API solubility.

Solubility of bulk APL-101 as received API was conducted in various biorelevant media. Small amounts of API were suspended in media and continuously agitated at room temperature for a period up to 24 hours. Samples were centrifuged to pellet out undissolved solids and the resulting supernatant was sampled, diluted, and analyzed by HPLC utilizing the short-assay method sued for dissolution sample analysis.

The kinetic solubility and sustainment of the API was measured in the presence of various polymer excipients that might serve as dispersion polymers in an amorphous solid dispersion (see EXAMPLE 1). The measured concentrations are compared to amorphous drug (dosed without any polymer presence in FaSSIF) at 5, 15, 30, 45 and 60 minutes, with a dose of 500 μgA/mL (micrograms active per mL). APL without polymer showed an initial peak concentration of 200 μgA followed by rapid precipitation to a low level. Good sustainment nearly at dose throughout the experiment was observed for 20:80 HPMCAS-H, other grades of HPMCAS and HPMC-E3LV showed some sustainment but precipitated prior to experiment end. Increased drug loading resulted in poorer performance, with all polymers showing rapid precipitation after an initial peak, however 20:80 APL-101:HPMCAS-H did sustain for 45 mins prior to precipitating. Results indicate a highly favorable interaction between APL-101 and HPMCAS-H, and that lower drug loadings will likely be needed to stabilize the API and prevent crystallization.

Patheon performed molecular modeling activities utilizing the Quadrant 2® platform to evaluate specific drug-drug and drug-polymer interactions for APL-101. Modeling methods ranged from high level quantum mechanics calculations to molecular mechanics and molecular dynamics using a suite of programs assembled by Patheon. The goals of this work were to examine the drug-drug and drug-polymer molecular level interactions between APL-101 and compendial GRAS polymers in order to provide a rational basis for selection of appropriate polymers for inclusion in a solubilized drug product intermediate. This rationale is based on molecular descriptors and specific drug-polymer interaction energies.

From the in silico modelling, APL-101 was determined to have favorable interactions with HPMCAS, HPMC, PVP VA64, PVP, HPMCP HP-55 and Eudragit L100-55. MDSC experiments of APL-101 provided a Tm/Tg ratio (K/K) of 1.37, indicative of moderate physical stability and a low propensity to crystallize from the dispersion. Based on historical Tm/Tg ratio experience, in silico molecular dynamics interactions, and in vitro solvent shift assay, SDI formulations at 20% and 33% drug loading with HPMCAS-H, HPMCAS-M, PVP-K30, and Eudragit L100-55 were nominated for manufacturing.

### Example 3

This example illustrates the focused screening of polymers used for the manufacture of solid dispersion.

Spray Dried Formulation Manufacturing

Eight APL-101:polymer dispersion formulations were chosen for feasibility screening at a batch size of 3 g APL-101. These formulations were spray dried from 80:20 DCM:MeOH. A secondary tray drying process was used to remove residual solvent after the initial spray drying process. In this operation, the “wet” SDI was heated to 45° C. and stored in a convection tray oven for roughly 24 hours. GC-HS was used to measure the residual solvent remaining from APL-101 SDI material after secondary drying. The 33:67 APL-101:PVP-K30 formulation required an additional 18 hours of drying. The residual solvent in all other formulations was below the MeOH limit (3000 ppm) and DCM limit (600 ppm) set forth by the International Conference on Harmonization (ICH).

Feasibility SDI Characterization

Initial feasibility SDI formulations were characterized by XRPD, SEM, MDSC, and biorelevant dissolution tests.

Thermal analysis by MDSC showed that all dispersions have a single Tg, indicating an intimately mixed amorphous solid dispersion with good homogeneity (Table 2). The non-reversing heat signal did not show crystallization or other events and has been omitted. These relatively high glass transition temperatures are an indication of good physical stability, i.e. the propensity of the API to crystallize during long-term storage is low. To ensure long-term physical stability, an SDI should be stored well below the Tg at a given condition so that mobility of the drug in the glass dispersion is very low.

TABLE 2
MDSC data of APL-101 Feasibility SDIs

| Formulations | Measured Tg (° C.) |
|---|---|
| 20:80 APL-101:HPMCAS-M SDI | 97 |
| 33:67 APL-101:HPMCAS-M SDI | 92 |
| 20:80 APL-101:HPMCAS-H SDI | 96 |
| 33:67 APL-101:HPMCAS-H SDI | 91 |
| 20:80 APL-101:PVP-K30 SDI | 140 |
| 33:67 APL-101:PVP-K30 SDI | 128 |
| 20:80 APL-101:HPMC E3LV SDI | 107 |
| 33:67 APL-101:HPMC E3LV SDI | 100 |

Initial characterization by XRPD indicates that the SDIs are amorphous dispersions and no crystalline peaks were observed in the SDI diffractograms.

Surface morphology of the SDI particles was characterized using scanning electron microscopy. Typical SDI morphology was observed consisting of whole and collapsed spheres with smooth surfaces, some shattered spheres were observed. No crystalline material was observed in any samples.

The dissolution performance of the feasibility SDIs and bulk APL-101 was tested in a biorelevant non-sink dissolution experiment as described in EXAMPLE 1. The design of this experiment is to rank order and select lead formulations. APL-101 bulk API showed very low dissolution performance in gastric and intestinal media. All SDI formulations provided an increase in drug dissolution and sustainment in intestinal media, with 20% APL-101:HPMCAS-H showing the highest dissolution performance with good sustainment throughout the experiment. Interestingly, the two HPMCAS-M SDIs showed higher initial Cmax in FaSSIF but quickly began to precipitate. Gastric solubility was consistently low among the SDIs, however significantly higher than crystalline API. 20% and 33% active HPMCAS-H and 20% active HPMC-E3LV and PVP-VA based formulations were nominated as the lead formulations. Dissolution results are shown in Table 3.

TABLE 3
Biorelevant Dissolution Data for APL-101
Feasibility SDIs Compared to Bulk APL-101

| | 1Cmax | | 3AUC35-210 |
|---|---|---|---|
| | FaSSIF | 2C210 | FaSSIF |
| Formulation | (μgA/mL) | (μgA/mL) | (min*μgA/mL) |
| 20:80 APL-101:HPMCAS-M | 290.2 | 87.1 | 26800 |
| SDI | | | |
| 33:67 APL-101:HPMCAS-M | 249.1 | 68.9 | 20700 |
| SDI | | | |
| 20:80 APL-101:HPMCAS-H | 212.6 | 191.8 | 35000 |
| SDI | | | |
| 33:67 APL-101:HPMCAS-H | 195.9 | 75.8 | 27600 |
| SDI | | | |
| 20:80 APL-101:PVP-K30 SDI | 51.2 | 22.0 | 5300 |
| 33:67 APL-101:PVP-K30 SDI | 77.9 | 25.5 | 6100 |
| 20:80 APL-101:HPMC E3LV | 59.5 | 52.8 | 9600 |
| SDI | | | |
| 33:67 APL-101:HPMC E3LV | 57.5 | 52.9 | 9600 |
| SDI | | | |
| APL-101 Form B | 8.9 | 7.8 | 1400 |

Lead formulations were characterized for chemical and physical stability during an accelerated stability study, but prior to initiation are tested by MDSC for glass transition suppression as a function of relative humidity.

Effect of Relative Humidity on Suppression of Glass Transition Temperature

The physical stability of the lead SDIs was evaluated by measuring the Tg at elevated humidity (32.8%, 50%, and 75.3% RH) conditions. Samples were stored at the elevated humidity conditions in saturated salt solutions for 18 hours at ambient temperature before analysis. Results are reported as a function of relative humidity (RH) in Table 4. All lead SDI formulations have a Tg that is low at elevated humidity conditions and is predicted to require conservative packaging (i.e. desiccant, foil-foil seal, or etc.) to obtain sufficient long-term physical stability of the SDI. To ensure long-term physical stability in open packaging at all ICH conditions, it is desirable that the SDI have a Tg higher than 50° C. at 75% RH, and ideally higher than 60° C. at 75% RH. Note that the 33:67 APL-101:PVP K30 SDI used in this study is the familiarization spray outlined in section 3.3, not the feasibility SDI described in this section.

TABLE 4
Tg as a Function of % RH for APL-101 Lead SDI Formulations

| | Tg at RH (° C.) | | |
|---|---|---|---|
| Sample | 32.8% | 50.0% | 75.3% |
| 20:80 APL-101:HPMCAS-H SDI | 76 | 68 | 45 |
| 33:67 APL-101:HPMCAS-H SDI | 71 | 67 | 41 |
| 33:67 APL-101 HPMC E3LV SDI | 69 | 55 | 27 |
| 33:67 APL-101:PVP-K30 SDI | 71 | 44 | 15 |

Feasibility SDI Accelerated Stability

To rapidly assess the physical and chemical stability of the lead APL-101 SDI formulations, the dispersions were aged for 3 weeks at 25° C./60% RH in open packaging, and 40° C./75% RH in open and closed packaging per stability protocol RD-ST-19-960-01. The SDIs were evaluated for physical and chemical stability by appearance, amorphous character by XRPD, assay and impurities by HPLC and particle morphology by SEM.

Appearance testing results differed by polymer. For both HPMCAS-H formulations the samples remained white powders at all conditions. At 40/75 open condition the HPMC-E3 SDI became clumpy, and the PVP-K30 SDI became a hard orange resin. This physical instability was expected based on polymer hygroscopicity and the Tg vs. % RH data collected previously. Visual observations are described below in Table 5.

TABLE 5
Visual Appearance of APL-101 Lead SDIs after 3 weeks Stability

| Sample Description | Storage Condition | Storage Time | Appearance |
|---|---|---|---|
| 20:80 APL- | NA | t = 0 | White Powder |
| 101:HPMCAS-H SDI | 25° C./60% RH/Open | 3 weeks | White Powder |
| | 40° C./75% RH/Closed | 3 weeks | White Powder |
| | 40° C./75% RH/Open | 3 weeks | White Powder |
| 33:67 APL- | t = 0 | t = 0 | White Powder |
| 101:HPMCAS-H SDI | 25° C./60% RH/Open | 3 weeks | White Powder |
| | 40° C./75% RH/Closed | 3 weeks | White Powder |
| | 40° C./75% RH/Open | 3 weeks | White Powder |
| 33:67 APL-101 HPMC | t = 0 | t = 0 | White Powder |
| E3LV SDI | 25° C./60% RH/Open | 3 weeks | White Powder |
| | 40° C./75% RH/Closed | 3 weeks | White Powder |
| | 40° C./75% RH/Open | 3 weeks | Clumpy White Powder |
| 33:67 APL-101 PVP-K30 | t = 0 | t = 0 | White Powder |
| SDI | 25° C./60% RH/Open | 3 weeks | Clumpy White Powder |
| | 40° C./75% RH/Closed | 3 weeks | Clumpy White Powder |
| | 40° C./75% RH/Open | 3 weeks | Orange resin |

SEM testing of the aged SDI samples showed no change in morphology after 3 weeks, with the exception of 33:67 APL-101:PVP-VA which consisted of API-like structures on large blocks of resin.

XRPD analysis of the aged SDI samples shows that all SDI formulations remained amorphous with no detectable crystalline material after 3 weeks with the exception of the PVP-K30 SDI. That sample had to be ground up by mortar and pestle for analysis and the large pieces resulted in the broader peaks that align fairly well with bulk APL-101 Form B.

Assay and impurities analysis of the aged SDI samples showed no change in related impurities relative to as-received bulk API in all samples after 3 weeks.

### Example 4

This example illustrates the characterization of the familiarization SDI by XRPD, SEM, MSDC, particle size distribution (PSD) by laser diffraction, and biorelevant dissolution tests.

Thermal analysis done by MDSC showed that the SDI has a single Tg of 126° C., indicating an intimately mixed amorphous solid dispersion with good homogeneity.

Characterization by XRPD indicates that the Familiarization SDI is an amorphous dispersion and no crystalline peaks were observed in the diffractogram.

Surface morphology of the familiarization SDI particles was characterized using scanning electron microscopy. Typical SDI morphology was observed consisting of whole and collapsed spheres with smooth surfaces. No crystalline material was observed in any sample.

The PDS of the familiarization SDI was determined by laser diffraction using a matersizer 3000 with a dry dispersant Aero S unit (Malvern Instruments). A Gaussian distribution was observed, with a Dv (50) of 3.0 m.

### Example 5

This example illustrates the manufacture and characterization of prototype SDI for APL-101.

After characterization and accelerated stability studies of the feasibility SDIs was completed, HPMCAS-H was chosen as the lead polymer for PK scale-up activities. The 33:67 APL-101:HPMCAS-H SDI formulation was chosen to allow for a higher overall dose in tablets.

The Prototype SDI formulation was characterized by XRPD, SEM, MSDC, and biorelevant dissolution.

Thermal analysis done by MDSC showed that the dispersion had a single Tg of 89° C., indicating an intimately mixed amorphous solid dispersion with good homogeneity and comparable to the feasibility SDI.

Characterization by XRPD indicates that the Prototype SDI is an amorphous dispersion and no crystalline peaks were observed in the diffractogram.

Surface morphology of the Prototype SDI particles was characterized using scanning electron microscopy. Typical SDI morphology was observed consisting of whole and collapsed spheres with smooth surfaces. No crystalline material was observed in any sample.

The PSD of the Prototype SDI was determined by laser diffraction as previously described. A distribution with a Dv(50) of 17.0 m was observed, significantly larger than the familiarization spray due to a different formulation and much higher % total solids in the spray solution.

The dissolution performance of the Prototype SDIs was tested in a biorelevant non-sink dissolution experiment (Table 6). The Prototype SDI showed similar dissolution performance to the feasibility SDI when test within several days of manufacture. However, during this body of work, the previously manufactured 20:80 APL-101:HPMCAS-H SDI was tested after aging for three months at ambient conditions, and the dissolution performances shows a significant decrease after transferring to intestinal media. The root cause of this decrease is unknown, but it is suspected to be related to either hydrate formation or compression. To investigate the compression effect on SDI and subsequent tablet biorelevant dissolution performance, the SDI for tablets, 33:67 APL-101:HPMCAS-H, was compressed as SDI and analyzed by biorelevant dissolution, the results of which show a similar decrease to the aged 20:80 APL-101:HPMCAS-H SDI, indicating that compression also plays a role in the decrease in dissolution performance.

TABLE 6
Biorelevant Dissolution Data for APL-101
Prototype SDI Compared to Bulk APL-101

| | Cmax FaSSIF | C210 | AUC 35-210 FaSSIF |
|---|---|---|---|
| Formulation | (μgA/mL) | (μgA/mL) | (min*μgA/mL) |
| 33:67 APL-101:HPMCAS-H SDI | 201.6 | 81.5 | 266600 |
| 33:67 APL-101:HPMCAS-H | 99.4 | 35.6 | 9100 |
| compressed SDI | | | |
| 20:80 APL-101:HPMCAS-H SDI | 212.6 | 191.8 | 35000 |
| 20:80 APL-101:HPMCAS-H SDI | 107.9 | 31.8 | 7800 |
| aged 3 months ambient | | | |
| APL-101 API | 8.9 | 7.8 | 1400 |

The results showed that SDI exhibits very significant improved in vitro dissolution performance relative to bulk crystalline free base material through relevant time-scale. The formulation demonstrated good physical and chemical stability, and with moisture protective packaging is expected to have an adequate shelf life stored ambient. The formulations can be manufactured and scaled-up using standard spray drying techniques and equipment. Although changed in SDI dissolution behavior were observed based on sample age and compression, the increase in AUC relative to crystalline API even after the decrease, is still quite significant. Based on the physiochemical characterization and biorelevant dissolution performance testing of the SDIs, enhanced in vivo exposure is expected for APL-101 utilizing a spray dried dispersion process.

### Example 6

This example illustrates the pK study of APL-101 table and capsule in Dog.

The study compared tablet and capsule oral formulations' systemic exposure in dog after single oral dosing. In general, male Beagle dogs of about 14 kg body weight were dosed with tablet or capsule of 100 mg. The PK was collected after 0.5, 1, 2, 4, 8, 24, 48 hours after dosing and subject to LC MS-MS analysis.

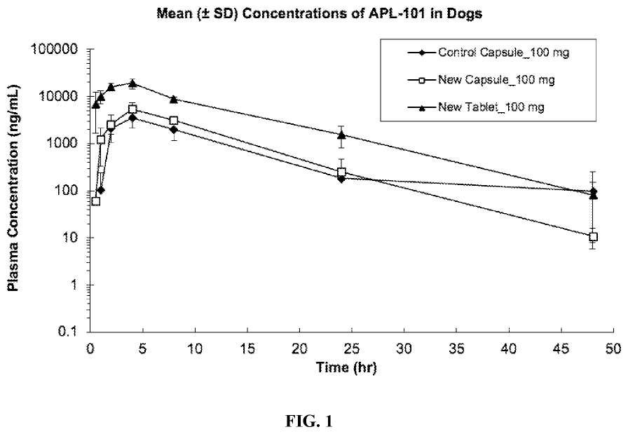

As shown in FIG. 1 and Table 7, tablet exhibited a 5 times more exposure than capsule.

TABLE 7
Systemic exposure of APL-101 in dog

| | | | Cₘₐₓ ± SD | AUCₗₐₛₜ ± SD |
|---|---|---|---|---|
| Treatment | t_{1/2} (hr) | tₘₐₓ (hr) | (ng/mL) | (hr*ng/mL) |
| Control | 8.76 | 3.33 | 3523 ± 1375 | 38441 ± 12172 |
| (Current Capsule) | | | (39.0%) | (31.7%) |
| New Capsule | 4.90 | 4.00 | 5413 ± 2028 | 57441 ± 11684 |
| | | | (37.5%) | (20.3%) |
| Tablet | 5.63 | 4.00 | 19067 ± 4726 | 212216 ± 49596 |
| | | | (24.8%) | (23.4%) |

What is claimed is:

1. A pharmaceutical composition comprising a formulation comprising an active pharmaceutical ingredient (API) and a polymer, wherein said API is a compound of the following formula or a pharmaceutically acceptable salt thereof or a hydrate thereof: 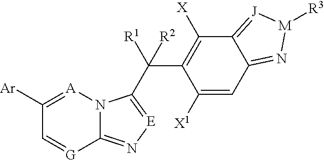
wherein:
R¹ and R² are independently hydrogen or halogen;
X and X¹ are independently hydrogen or halogen;
A and G are independently CH or N, or CH=G is replaced with a sulfur atom;
E is N;
J is CH, S or NH;
M is N or C;
Ar is aryl or heteroaryl, optionally substituted with 1-3 substituents independent selected from: C₁₋₆alkyl, C₁₋₆alkoxyl, halo C₁₋₆alkyl, halo C₁₋₆alkoxy, C₃₋₇cycloalkyl, halogen, cyano, amino, —CONR⁴R⁵, —NHCOR⁶, —SO₂NR⁷R⁸, C₁₋₆alkoxyl-, C₁₋₆alkyl-, amino-C₁₋₆alkyl-, heterocyclyl and heterocyclyl-C₁₋₆alkyl-, or two connected substituents together with the atoms to which they are attached form a 4-6 membered lactam fused with the aryl or heteroaryl;
R³ is hydrogen, C₁₋₆alkyl, C₁₋₆alkoxy, haloC₁₋₆alkyl, halogen, amino, or —CONH—C₁₋₆alkyl-heterocyclyl;
R⁴ and R⁵ are independently hydrogen, C₁₋₆alkyl, C₃₋₇cycloalkyl, heterocyclyl-C₁₋₆alkyl, or R⁴ and R⁵ together with the N to which they are attaches form a heterocyclyl;
R⁶ is C₁₋₆alkyl or C₃₋₇cycloalkyl;
R⁷ and R⁸ are independently hydrogen or C₁₋₆alkyl; and
wherein said polymer is selected from the group consisting of poly(vinylpyrrolidone)-co-vinyl acetate (PVP-VA 64), hydroxypropyl methylcellulose (HPMC), poly(vinylpyrrolidone) (PVP), hydroxypropyl methylcellulose acetate succinate (HPMCAS), poly(methacrylic acid-co-methyl methacrylate) 1:1 (Eudragit L 100), hydroxypropyl methylcellulose phthalate (HPMCP-HP55) and a combination thereof.

2. The pharmaceutical composition of claim 1, wherein the API is selected from the group consisting of

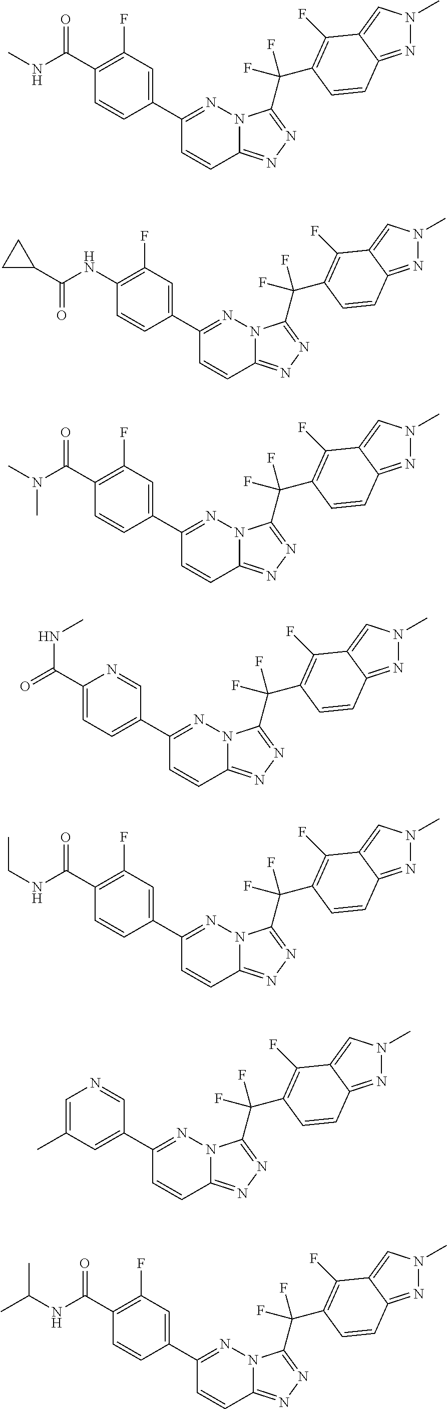

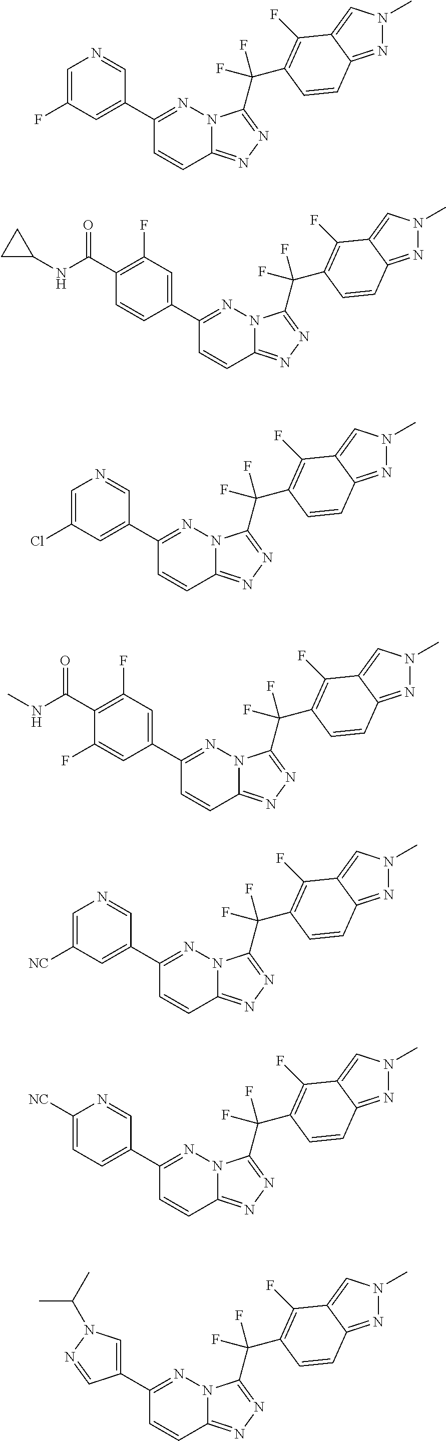

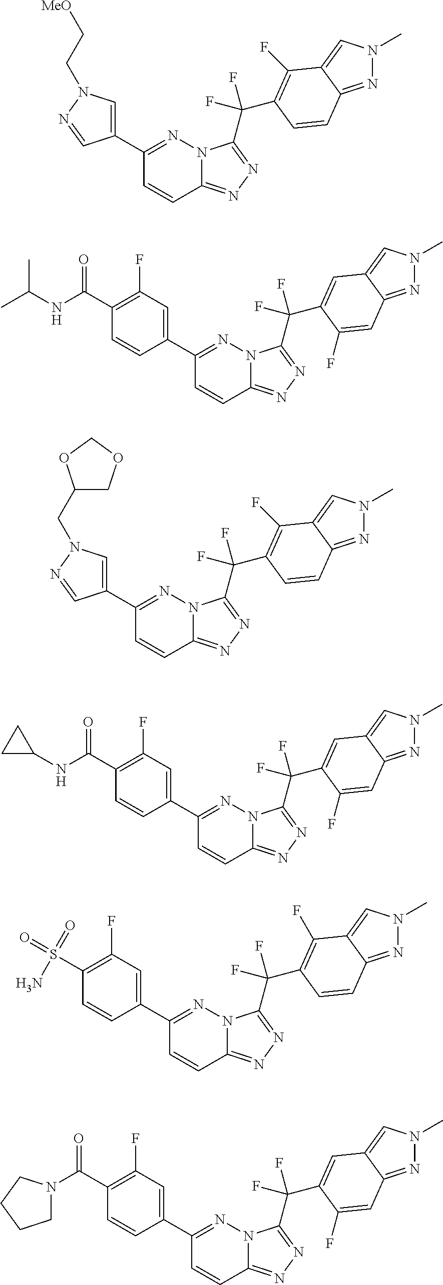

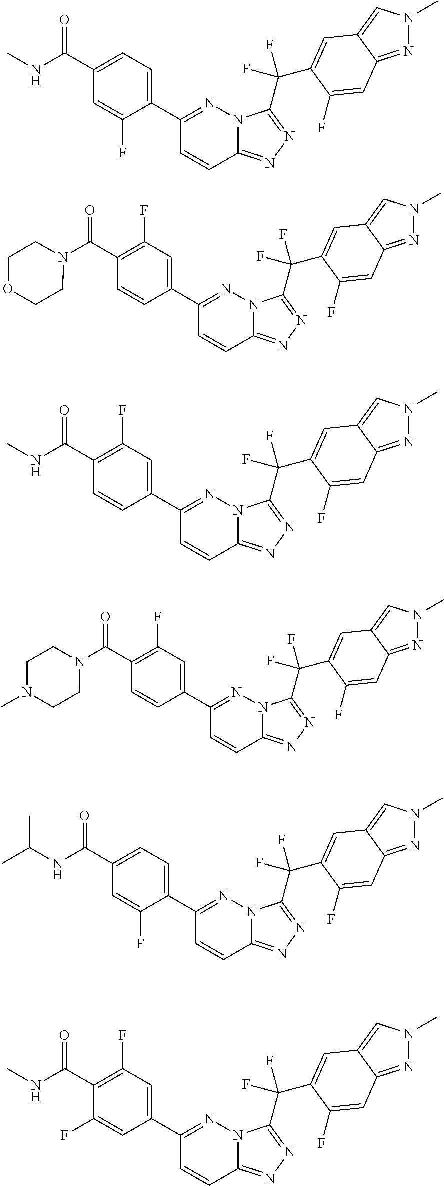

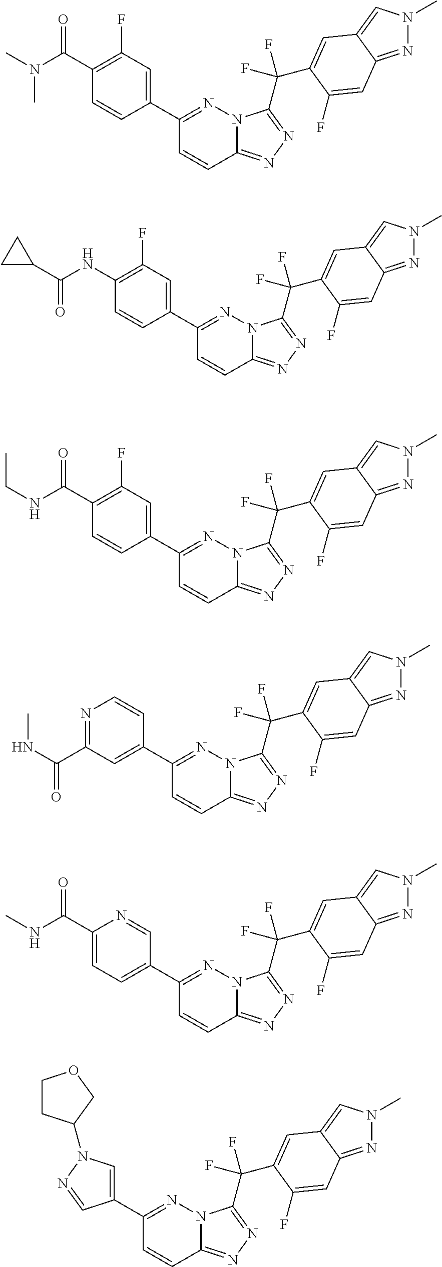

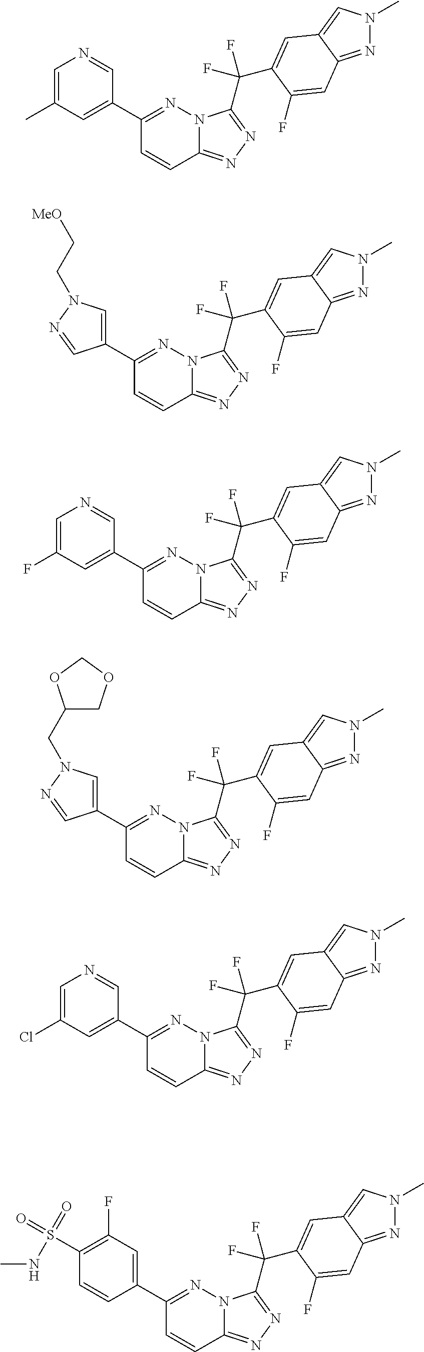

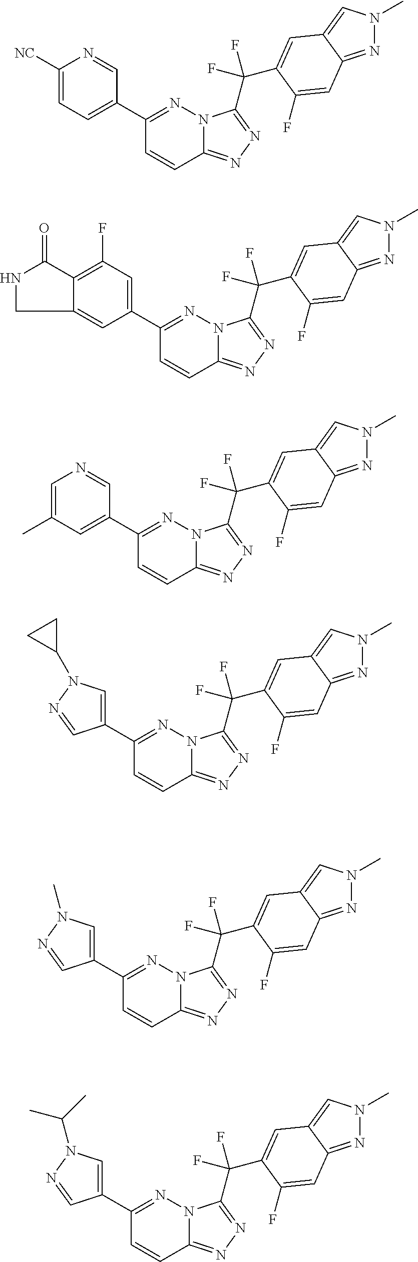

3. The pharmaceutical composition of claim 1, wherein the API has a formula of

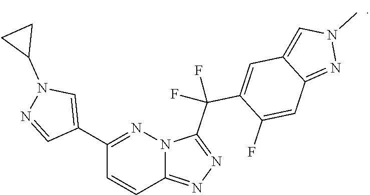 [SMILES: Cn1cc2cc(C(F)(F)c3nnc4ccc(-c5cnn(C6CC6)c5)nn34)c(F)cc2n1]

4. The pharmaceutical composition of claim 1, wherein the API has a weight percentage of 10-40% in the formulation.

5. The pharmaceutical composition of claim 1, wherein the API has a weight percentage of 20-33% in the formulation.

6. The pharmaceutical composition of claim 1, wherein the polymer is HPMCAS.

7. The pharmaceutical composition of claim 6, wherein the polymer is HPMCAS-H.

8. The pharmaceutical composition of claim 1, wherein the formulation is a solid dispersion.

9. The pharmaceutical composition of claim 8, wherein the solid dispersion is prepared by spray drying.

10. The pharmaceutical composition of claim 1, which is an oral dosage form.

11. The pharmaceutical composition of claim 1, which is a tablet.

12. A method for treating cancer in a subject, the method comprising administering to the subject a pharmaceutical oral dosage form of claim 1.

13. The method of claim 12, wherein the cancer is selected from the group consisting of lung cancer, melanoma, renal cancer, liver cancer, myeloma, prostate cancer, breast cancer, colorectal cancer, pancreatic cancer, thyroid cancer, hematological cancer, leukemia and non-Hodgkin's lymphoma.

14. The method of claim 13, wherein the cancer is non-small cell lung cancer (NSCLC) or hepatocellular carcinoma.

15. A pharmaceutical composition comprising a formulation comprising an active pharmaceutical ingredient (API) and a polymer, wherein said API is 6-(1-cyclopropylpyrazol-4-yl)-3-[difluoro-(6-fluoro-2-methylindazol-5-yl)methyl]-[1,2,4]triazolo[4,3-b]pyridazine (APL-101), which has the following formula, or a pharmaceutically acceptable salt thereof or a hydrate thereof: 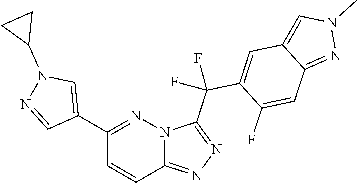 [SMILES: Cn1cc2cc(C(F)(F)c3nnc4ccc(-c5cnn(C6CC6)c5)nn34)c(F)cc2n1]
wherein said polymer is selected from the group consisting of poly(vinylpyrrolidone)-co-vinyl acetate (PVP-VA 64), hydroxypropyl methylcellulose (HPMC), poly(vinylpyrrolidone) (PVP), hydroxypropyl methylcellulose acetate succinate (HPMCAS), poly(methacrylic acid-co-methyl methacrylate) 1:1 (Eudragit L 100), hydroxypropyl methylcellulose phthalate (HPMCP-HP55) and a combination thereof.
